(19) United States
(12) Patent Application Publication
(10) Pub. No.: US 2009/0318897 A1
(43) Pub. Date: Dec. 24, 2009

(54) GASTROJEJUNAL FEEDING ASSEMBLY

(75) Inventors: F. Kristen Bailey, Bloomington, IN (US); Jeffry S. Melsheimer, Springville, IN (US)

Correspondence Address: BRINKS HOFER GILSON & LIONE/INDY/COOK;BRINKS HOFER GILSON & LIONE, CAPITAL CENTER, SUITE 1100, 201 NORTH ILLINOIS STREET, INDIANAPOLIS, IN, 46204-4220, US

(73) Assignee: Cook Critical Care Incorporated, Bloomington, IN (US)

(21) Appl. No.: 12/143,278

(22) Filed: Jun. 20, 2008

(51) Int. Cl. A61M 25/14; A61M 25/098; A61M 25/02

(52) U.S. Cl. 604/537; 604/539; 604/529

(57) ABSTRACT

An assembly for delivering nutritional products to the jejunum of a patient through a stoma formed through the abdominal wall and the stomach wall. A first tubular member is alignable such that the proximal end communicates with an area exterior of the patient, and the distal end is disposed within an interior space of the stomach of the patient. At least a portion of the distal end within the stomach interior space is configured for maintaining the stomach wall in apposition with the abdominal wall. A second tubular member is alignable such that the proximal end communicates with an area exterior of the patient, and the distal end extends into the jejunum of the patient. A hub positioned in the stoma has a first opening communicating with the first tubular member, and a second opening communicating with the second tubular member. Respective closure members may be provided for selectively sealing the first and second openings.

### BACKGROUND OF THE INVENTION

Technical Field. The present invention relates to medical feeding devices, and more particularly, to an assembly and method for transmitting nutritional products to the jejunum of a patient.

Background Information. Patients for whom normal ingestion of food becomes difficult or impossible may require placement of a feeding tube to assist in providing their nutritional needs. For some individuals, such as comatose patients, stroke victims, or those with a compromised gastrointestinal tract, this may require placement of a tube that is introduced percutaneously into the stomach for delivery of nutritional products directly into the stomach. The nutritional products are delivered to the interior of the stomach through the distal end of the tube and/or through side ports along the length of the tube. Tubes for delivery of nutritional products into the stomach are generally referred to as gastrostomy tubes, or “G”-tubes.

Due to the existence of congenital abnormalities and other factors, such as severe gastric reflux and a high aspiration risk, some patients are not good candidates for feeding into the stomach through a G-tube. In such patients, feeding may often be accomplished by inserting a feeding tube, referred to as a jejunostomy tube, or a “J”-tube, that extends on through the stomach and directly into the jejunum (the middle section of the small intestine). The J-tube bypasses the stomach, thereby decreasing the risk of gastric reflux and aspiration. In many cases, a J-tube may be inserted into the jejunum through the interior of a G-tube that has been previously positioned in the stomach, e.g., via the Seldinger percutaneous entry technique.

Although J-tubes have been found to be generally efficient in providing nutrition to a patient in need of the same, there are some difficulties associated with the use of such tubes. For example, due to the wide range of differences in thicknesses of the abdominal and/or stomach wall between patients, a “one-size-fits-all” device may not be effective, or possible, for use with all patients. In addition, since the jejunum is generally offset relative to the stomach, it can be difficult to properly direct the end of a J-tube into the jejunum, and particularly, past the ligament of Treitz. Also, J-tubes are generally very flexible, which contributes to this difficulty in directing the tubes to the desired area. Further, once they are properly positioned, J-tubes may be subject to dislodgement.

It would be desirable to provide a feeding assembly for providing nutritional products directly into the jejunum of a patient, which feeding assembly is capable of self-adjustment for use with patients having a wide range of abdominal and stomach wall thicknesses.

### SUMMARY

The problems of the prior art are addressed by the features of the present invention. In one form thereof, the invention comprises an assembly for delivery of nutritional products to the jejunum of a patient through a stoma formed in the abdominal wall and the stomach wall of the patient. A first tubular member has a proximal end and a distal end. The first tubular member is sized and alignable such that the proximal end communicates with an area exterior of the patient, and the distal end is disposed within an interior space of the stomach of the patient. At least a portion of the distal end within the stomach interior space is configured for maintaining the stomach wall in substantial apposition with the abdominal wall. A second tubular member has a proximal end and a distal end. The second tubular member is sized and alignable such that the proximal end communicates with an area exterior of the patient and the distal end substantially extends into the jejunum of the patient. A hub has a plurality of sealable openings, wherein a first opening communicates with the first tubular member, and a second opening communicates with the second tubular member. A first closure member is sized and arranged for selectively sealing the first opening, and a second closure member is sized and arranged for selectively sealing the second opening.

In another form thereof, the invention comprises a method for delivering nutritional products to the jejunum of a patient. A stoma is formed that extends through the abdominal wall and the stomach wall of a patient. A feeding assembly for delivery of the nutritional products to the jejunum is provided. The feeding assembly includes a first tubular member having a proximal end and a distal end. The first tubular member is sized and alignable such that the proximal end communicates with an area exterior of the patient, and the distal end is insertable into an interior space of the stomach of the patient. At least a portion of the first distal end is manipulatable from a generally elongated condition to a condition configured for maintaining the stomach wall in substantial apposition with the abdominal wall. A second tubular member has a proximal end and a distal end. The second tubular member is sized and alignable such that the proximal end communicates with an area exterior of the patient, and the distal end is extendable into the jejunum of the patient. A hub has a plurality of openings, wherein a first opening communicates with the first tubular member, and a second opening communicates with the second tubular member. The distal ends of the first and second tubular members are inserted through the stoma into the interior space of the stomach, the distal end of the first tubular member being inserted in the generally elongated condition. The distal end of the first tubular member is manipulated from the generally elongated condition to the condition for maintaining the stomach wall in substantial apposition with the abdominal wall. The distal end of the second tubular member is advanced through the duodenum into the jejunum of the patient.

In yet another form thereof, the invention comprises an assembly for delivery of nutritional products to a patient. The assembly comprises a first tubular member having a proximal end and a distal end. The first tubular member is sized and alignable such that the proximal end communicates with an area exterior of the patient, and the distal end is disposed within an interior space of the stomach of the patient. At least a portion of the distal end within the stomach interior space is configured for maintaining the stomach wall of the patient in substantial apposition with the abdominal wall. A second tubular member has a proximal end and a distal end. The second tubular member is sized and alignable such that the proximal end communicates with an area exterior of the patient and the distal end extends into the jejunum of the patient. A hub has a plurality of openings, wherein a first opening communicates with the first tubular member, and a second opening communicates with the second tubular member.

### BRIEF DESCRIPTION OF THE DRAWINGS

FIG. 1 is a perspective view of a gastrojejunal feeding assembly according to an embodiment of the present invention, with the closure members in an open position;

FIG. 2 is a is a perspective view of the gastrojejunal feeding assembly as shown in FIG. 1, with the closure members arranged to close the hub openings;

FIG. 3 is a view of the proximal end of the gastrojejunal feeding assembly of FIG. 1, showing the assembly situated such that the stomach wall and abdominal wall are held in apposition;

FIG. 4 is a view similar to that of FIG. 3, wherein the assembly is holding in apposition a stomach wall and abdominal wall of a thickness differing from that in FIG. 3;

FIG. 5 is a view similar to that of FIG. 3, showing the distal end of the feeding assembly extending into the jejunum; and

FIG. 6 is a view similar to FIG. 3 of the feeding assembly during initial introduction through the stoma, wherein a stylet is inserted through the lumen of tubular member 12 to straighten out the curls for insertion.

### DESCRIPTION OF PREFERRED EMBODIMENTS

For purposes of promoting an understanding of the present invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

In the following discussion, the terms “proximal” and “distal” will be used to describe the opposing axial ends of the inventive assembly, as well as the axial ends of various component features. The term “proximal” is used in its conventional sense to refer to the end of the assembly (or component thereof) that is closest to the operator during use of the apparatus. The term “distal” is used in its conventional sense to refer to the end of the assembly (or component thereof) that is initially inserted into the patient, or that is closest to the patient during use.

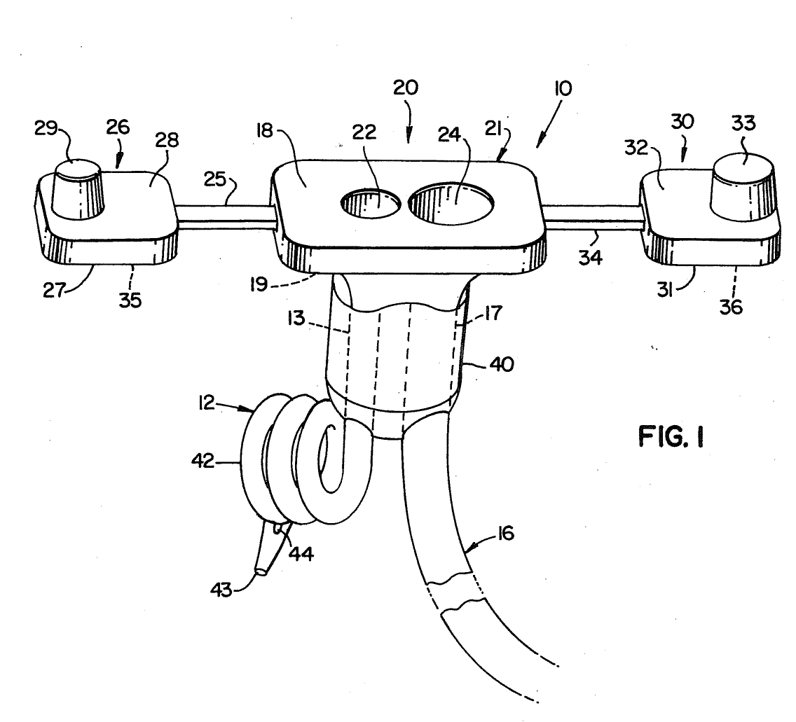

FIG. 1 illustrates a perspective view of a gastrojejunal (“GJ”) feeding assembly 10 in accordance with an embodiment of the present invention. GJ feeding assembly 10 includes a pair of tubular members 12, 16, and a hub 20 positioned to receive the respective proximal ends 13, 17 of tubular members 12, 16.

Hub 20 includes a main body portion 21 having an upper surface 18, a lower surface 19, and respective openings 22, 24 extending therethrough. Main body portion 21 may have any shape or configuration suitable for receiving tubular member proximal ends 13, 17 as shown, such that the interior of each of the tubular members 12, 16 communicates with an environment external of hub 20. Tube proximal ends 13, 17 may be integrally formed with main body portion 21, or alternatively, may be affixed to main body portion 21 at lower surface 19 by any conventional means, such as adhesion, heat-bonding, insert molding, etc.

Manipulatable closure members 26, 30 are provided for respective hub openings 22, 24. In the embodiment shown in FIG. 1, manipulatable closure members 26, 30 include respective base portions 27, 31. Base portion 27 has generally flat opposing surfaces 28, 35, and base portion 31 has generally flat opposing surfaces 32, 36, respectively. Respective stoppers 29, 33 sized and shaped for selectively sealing hub openings 22, 24 project from base portion flat surfaces 28, 32 in substantially perpendicular fashion. Flexible extensions 25, 34 connect respective closure members 26, 30 to hub main body portion 21.

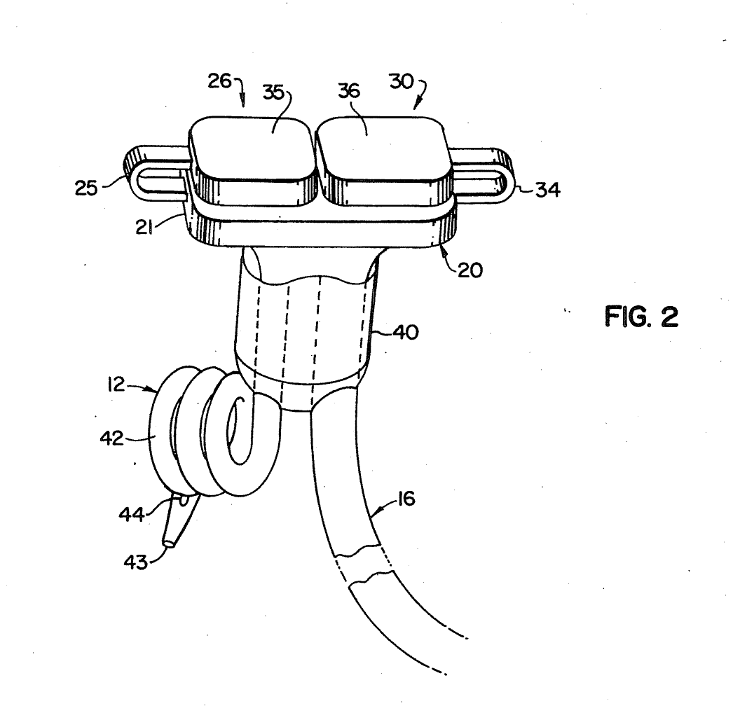

FIG. 2 is a perspective view of the gastrojejunal feeding assembly of FIG. 1. However in FIG. 2, flexible extension members 25, 34 are flexed, bent, or otherwise manipulated such that closure members 26, 30 are arranged in the closed position as shown. In this arrangement, stoppers 29, 33 are received in respective hub openings 22, 24 to seal the opening from passage of fluids therethrough.

Hub 20 may also include an optional generally cylindrical housing member 40. Housing member 40 extends distally from hub main body portion 21, and is sized and shaped to receive proximal ends 13, 17 of respective tubular members 12, 16. Generally, housing member 40 will be formed, e.g., by insert molding or other conventional method, to have separate channels therethrough (not shown) sized and shaped for receiving the respective tubular member proximal ends 13, 17.

Preferably, main body portion 21, manipulatable closure members 26, 30 and housing member 40 are formed from silicone rubber, polyurethane, or a co-polymer of the two. Other compositions having similar properties, such as ABS, urethane, and nylon may also be used. Flexible extensions 25, 34 are preferably formed from urethane or nylon.

In the embodiments shown, tubular member 12 is shaped such that its distal end comprises a plurality of wound portions, or curls 42. Curls 42 are preferably arranged in a generally helical fashion as shown in the figures, and are preferably arranged in the nature of coils of a spring. Preferably, curls extend laterally from tube proximal end 13 as shown, i.e., the long axis of the coil is perpendicular to the long axis of hub housing member 40. Alternatively, curls 42 can be arranged such that they extend distal to proximal end 13. Tubular member 12 may also be formed from a flexible and atraumatic material, such as silicone rubber, polyurethane, or a co-polymer of the two.

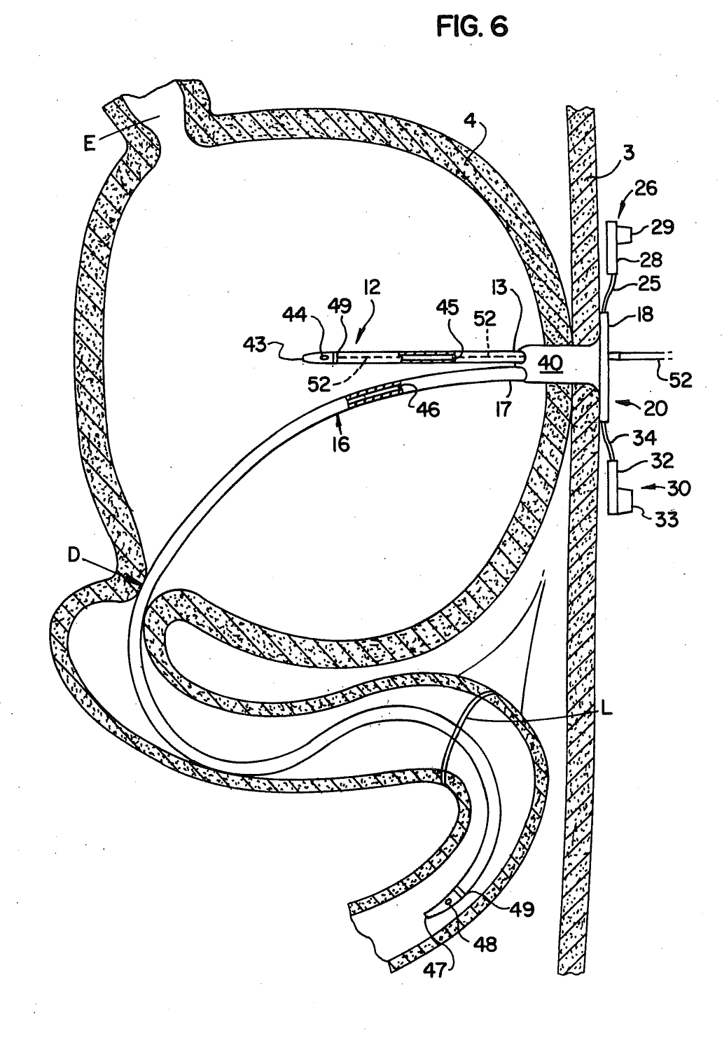

From the lateral configuration shown, curls 42 may be straightened into an elongated shape to ease insertion of the feeding assembly 10 through the abdominal wall of the patient. This arrangement is shown in FIG. 6. A stylet 52 is inserted into the lumen 45 of tubular member 12 through hub opening 22 for straightening curls 42 into the elongated condition shown. Tubular member 12 is formed to have an elasticity or shape memory so that curls 42 have a tendency to return to the coiled configuration upon removal of the stylet. Those skilled in the art are well aware of various means for imparting an internal memory to a flexible device of this type, e.g., by heat setting, and any conventional means of doing so may be employed.

Although the distal end of tubular member 12 is illustrated herein as an arrangement of curls, this is merely one example of a suitable configuration at the distal end. Those skilled in the art will appreciate that instead of the arrangement of curls 42 illustrated in FIG. 1, the distal end of tubular member 12 can alternatively be manipulated into any shape suitable for the purposes to be described herein. Thus, other configurations wherein a larger diameter lateral arrangement (e.g., curls 42) can be straightened into an elongated smaller diameter configuration (e.g., via introduction of the stylet), and then return to the original configuration may be substituted. When in an elongated condition, tubular member 12 essentially follows a relatively straight path in the stomach, similar to that of tubular member 16.

Preferably, lumen 45 (FIG. 6) extends the full length of the tubular member 12 to an open distal end 43. In addition, tubular member 12 may include one or more side ports 44 generally adjacent open distal end 43 and communicating with the lumen. Stomach contents may be aspirated through lumen 45 via distal end 43 and/or side port(s) 44, and thereafter out of assembly 10 through hub opening 22.

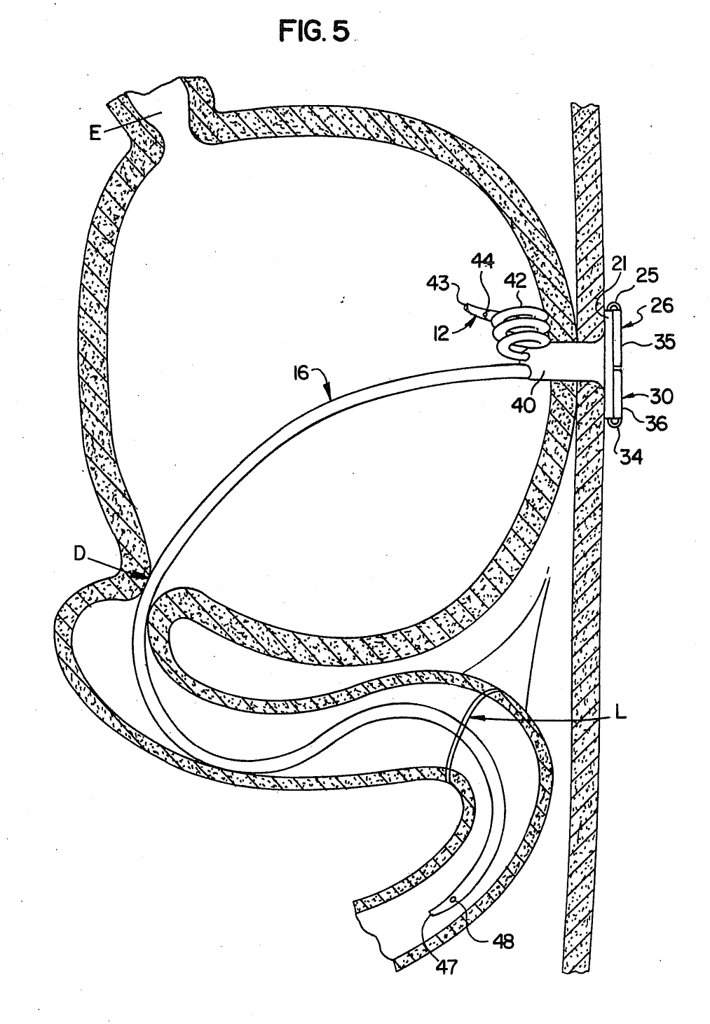

Tubular member 16 is sized to extend from hub 20 into the jejunum, distal to the ligament of Treitz. Generally speaking, the ligament of Treitz is considered the most cranial means of Mesenteric suspension before the small intestine changes direction, or descends. It creates a landmark, or an obstacle of sorts, that assists in assuring that sufficient purchase has been achieved. (FIG. 5). The position of the ligament of Treitz is generally indicated by reference element L in FIGS. 5 and 6. The length of tubular member 16 may vary between patients, but is typically between about 60 and 80 cm. Tubular member 16 has a full-length lumen 46 (FIG. 6) extending therethrough to open distal end 47. The tubular member may include one or more side ports 48 generally adjacent open distal end 47 and communicating with lumen 46 to provide additional openings for fluid transmission. Lumen 46 provides a pathway for the nutrients from a reservoir outside of the body through tubular member 16 to the jejunum. Preferably, tubular member 16 is also formed from a flexible and atraumatic material, such as silicone rubber, polyurethane, or a co-polymer of the two.

Tubular member 16 may have a diameter the same as, smaller than, or larger than, the diameter of tubular member 12. Typically, the tubular members will have a diameter between about 4.5 and 8.0 mm. It is generally preferred to provide tubular member 16 with a larger inner diameter than that of tubular member 12. Although tubular member 12 provides the physical structure when in the curled condition to maintain the abdominal wall 3 and stomach wall 4 in apposition, the lumen 45 extending through tubular member 12 will generally only need to have a large enough diameter to allow gases to pass through the tubular member. Lumen 46 of tubular member 16, on the other hand, should be large enough to allow nutritional products to pass through. Therefore, it is typically advantageous to provide lumen 46 with as large a diameter as reasonably possible. This is particularly important when it is desired to supply nutrients that may include suspended solid portions, in order to avoid clogging of the lumen.

If desired, tubular member 16 may be provided with features on the exterior surface of the tubular member to enable the tubular member to self-advance into the jejunum. Such features are known in the art, and are described, e.g., in U.S. Pat. Nos. 6,589,213 and 6,767,339, incorporated by reference herein. As disclosed in the incorporated-by-reference patents, the surface features, such as the series of cilia-like flaps shown and described in the '339 patent, could be positioned along the device in a manner such that naturally occurring peristaltic contractions grasp the surface structure of the tubular member upon insertion, and carry the tubular member in the direction of the jejunum. Self-advancing tubes, such as those described in the '339 patent, are available commercially from Cook Incorporated, of Bloomington, Ind., and are sold under the trademark TIGER TUBE®.

In addition to the foregoing, either or both of respective tubular members 12, 16 may be provided with features that promote visualization of the tube while in vivo. One such feature comprises a marker band 49 (FIG. 6) that can be provided at or near the distal end of the tubular member. Alternatively, a radiopaque filler can be incorporated into the matrix of the tubular member.

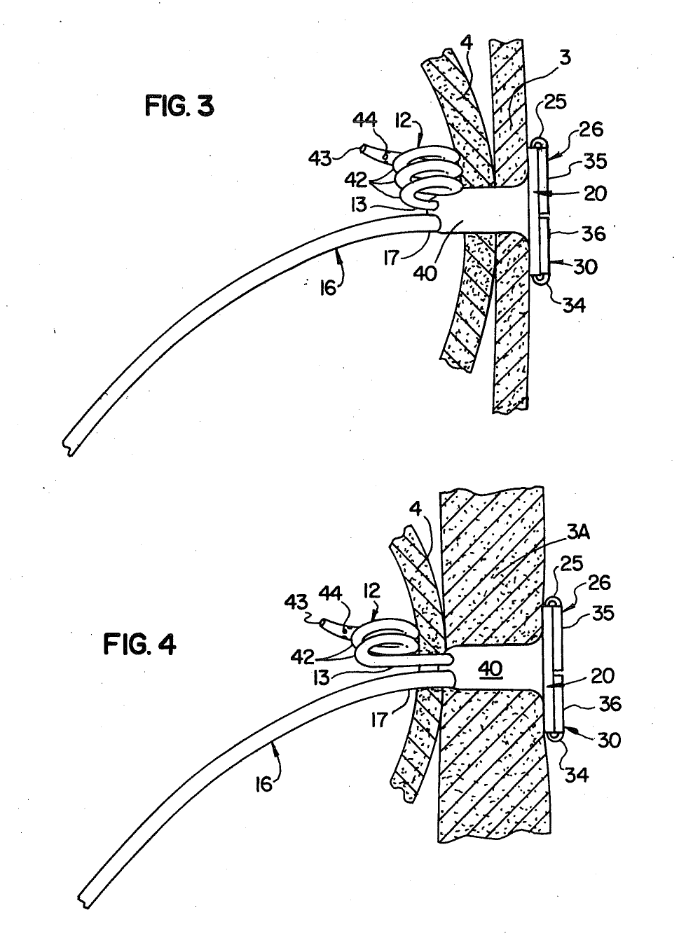

FIGS. 3 and 4 illustrate use of the inventive GJ feeding assembly 10 in two separate patients, having respective abdominal walls of different thicknesses. In FIG. 3, the patient has a relatively thin abdominal wall 3 and a stomach wall 4. In FIG. 4, the patient has a relatively thick abdominal wall 3A and a stomach wall 4. Other than the depiction of the abdominal wall in the respective figures by reference numerals 3 (in FIG. 3) and 3A (in FIG. 4), the remaining designations of corresponding elements in the two figures are the same.

The feeding assembly 10 may be inserted through a stoma formed through the abdominal wall 3 and stomach wall 4 of the patient. Initially, it is generally desired to insufflate the stomach by conventional means to bring the anterior stomach wall into apposition with the abdominal wall. One or more conventional T-fasteners (not shown) may be utilized to maintain this apposed arrangement of the stomach wall and abdominal wall. A needle may be used to puncture the apposed abdominal and stomach walls, and a wire guide may be inserted into the stomach through a bore of the needle utilizing, e.g., the well-known Seldinger percutaneous access technique. The opening may be dilated to the desired diameter utilizing an appropriately-sized dilator, or a series of dilators. An introducer sheath, such as a conventional splittable sheath, may be passed over the dilator and into the stoma formed thereby, and the dilator can be removed.

Stylet 52 is inserted into the lumen 45 of tubular member 12 (FIG. 6), and the inventive feeding assembly 10 is positioned for entry into the stoma. The respective distal ends of tubular members 12, 16 are passed through the introducer sheath such that they enter the interior of the stomach. Housing member 40, if present, or the proximal ends of tubular members 12, 16 if no housing member is present, is passed through the interior of the introducer sheath. The spliftable introducer sheath is then removed in conventional manner, and the housing member is seated in the stoma, as shown, e.g., in FIGS. 3-6.

Upon insertion of the distal end of feeding assembly 10 through the stoma, the stylet is thereafter removed. Upon removal of the stylet, the memory imparted to the distal end 13 of tubular member 12 results in the formation of curls 42. Curls 42 may be perhaps best visualized as the coils of a weak spring that is biased to curl up to the extent possible. Further “curling” of distal end 13 is limited by impact with the interior of stomach wall 4. As shown, curls 42 exert a clamping force against stomach wall 4, to maintain the stomach wall in apposition with abdominal wall 3.

Tubular member 12 can be structured to form as many curls as desirable to accommodate a range of clamping distances and clamping forces. As illustrated in FIG. 4, the presence of the thicker abdominal wall 3A reduces the amount of curling of distal end 13 required to position and maintain stomach wall 4 in apposition with abdominal wall 3A.

FIG. 5 illustrates the position of feeding assembly 10 when insertion is complete. Typically, the distal tip of tubular member 16 is directed into the duodenum “D” by the physician. Alternatively, the tubular member can be provided with structure, such as the cilia-like flaps shown in the incorporated-by-reference '339 patent and discussed above, to assist the tube in self-advancing through the duodenum in the direction of the jejunum. If visualization assistance for placement of the feeding assembly is desired, an endoscope can be passed into the stomach through esophagus “E”.

Although the invention has been described herein with reference to its primary intended use for delivering nutritional products to a patient in need of same as described, those skilled in the art will appreciate that additional features could be added to the assembly. For example, additional plugs could be provided, e.g., to seal gases and fluids, and to keep contaminants out. Similarly, the hub can be structured to accommodate discretely shaped (e.g., tapered) receptacles to accommodate standard enteric feeding equipment, and/or aspiration syringes. These, and other, routine modifications will be apparent to a skilled artisan in view of the teachings of the present invention.

While these features have been disclosed in connection with the illustrated preferred embodiments, other embodiments of the invention will be apparent to those skilled in the art that come within the spirit of the invention as defined in the following claims.

1. An assembly for delivery of nutritional products to the jejunum of a patient through a stoma formed in the abdominal wall and the stomach wall of the patient, comprising:
a first tubular member having a proximal end and a distal end, said first tubular member sized and alignable such that said proximal end communicates with an area exterior of the patient, and said distal end is disposed within an interior space of the stomach of the patient, at least a portion of said distal end within said stomach interior space configured for maintaining said stomach wall in substantial apposition with said abdominal wall;
a second tubular member having a proximal end and a distal end, said second tubular member sized and alignable such that said proximal end communicates with an area exterior of the patient and said distal end substantially extends into the jejunum of the patient;
a hub having a plurality of sealable openings, a first one of said openings communicating with said first tubular member, and a second one of said openings communicating with said second tubular member; and
a first closure member sized and arranged for selectively sealing said first opening, and a second closure member sized and arranged for selectively sealing said second opening.

2. The assembly of claim 1, wherein a length of said configured portion of said distal end of said first tubular member is variable during use of said assembly depending upon a dimension of at least one of said abdominal wall and said stomach wall.

3. The assembly of claim 1, wherein said hub comprises a first portion configured to be disposed exterior of the patient, and a second portion sized to at least substantially extend through said stoma and span said abdominal wall and said stomach wall.

4. The assembly of claim 1, wherein said hub further comprises a housing member extendable into said stoma, said first and second tubular members extending through said housing member.

5. The assembly of claim 1, wherein said first tubular member distal end comprises a curled portion.

6. The assembly of claim 5, wherein said first tubular member comprises a lumen extending therein, said assembly further comprising a stylet receivable within said lumen, said stylet capable of straightening said curled portion upon insertion into said lumen.

7. The assembly of claim 1, wherein at least one of said first and second tubular members includes a radiopaque marker band or a radiopaque filler.

8. The assembly of claim 1, wherein said hub comprises a main body portion having said openings extending therethrough, said first tubular member proximal end received in a first one of said openings, and said second tubular member proximal end received in a second one of said openings.

9. The assembly of claim 1, wherein each of said first and second tubular members has a lumen extending therethrough, said second tubular member lumen having a greater cross-section diameter than a cross-section diameter of said first tubular member lumen.

10. The assembly of claim 1, wherein said second tubular member has a lumen extending therethrough to an open distal end, and further includes at least one side port communicating with said lumen.

11. A method for delivering nutritional products to the jejunum of a patient, comprising:
forming a stoma that extends through the abdominal wall and the stomach wall of a patient;
providing a feeding assembly for delivery of said nutritional products to the jejunum, said feeding assembly comprising a first tubular member having a proximal end and a distal end, said first tubular member sized and alignable such that said proximal end communicates with an area exterior of the patient, and said distal end is insertable into an interior space of the stomach of the patient, at least a portion of said first distal end manipulatable from a generally elongated condition to a condition configured for maintaining said stomach wall in substantial apposition with said abdominal wall; a second tubular member having a proximal end and a distal end, said second tubular member sized and alignable such that said proximal end communicates with an area exterior of the patient and said distal end is extendable into the jejunum of the patient; and a hub having a plurality of openings, a first one of said openings communicating with said first tubular member, and a second one of said openings communicating with said second tubular member;
inserting said distal end of said first tubular member in said generally elongated condition, and said distal end of said second tubular member through said stoma into an interior space of said stomach;
manipulating the distal end of said first tubular member from said generally elongated condition to said condition for maintaining said stomach wall in substantial apposition with said abdominal wall; and
advancing the distal end of the second tubular member through the duodenum into the jejunum of the patient.

12. The method of claim 11, further comprising the step of insufflating the stomach to bring the stomach wall in apposition with the abdominal wall.

13. The method of claim 11, wherein said first tubular member distal end portion is manipulatable by inserting a stylet into a lumen of said first tubular member to achieve said generally elongated condition, and removing said stylet following insertion of said first tubular member distal end into said interior space of the stomach to achieve said condition configured to maintain said stomach wall in substantial apposition with said abdominal wall.

14. The method of claim 11, wherein said feeding assembly further includes a first closure member sized and arranged for selectively sealing said first opening, and a second closure member sized and arranged for selectively sealing said second opening.

15. The method of claim 11, wherein said hub further comprises a housing member extendable into said stoma, said first and second tubular members extending through said housing member.

16. An assembly for delivery of nutritional products to a patient, comprising:
a first tubular member having a proximal end and a distal end, said first tubular member sized and alignable such that said proximal end communicates with an area exterior of the patient, and said distal end is disposed within an interior space of the stomach of the patient, at least a portion of said distal end within said stomach interior space configured for maintaining the stomach wall of the patient in substantial apposition with the abdominal wall;
a second tubular member having a proximal end and a distal end, said second tubular member sized and alignable such that said proximal end communicates with an area exterior of the patient and said distal end extends into the jejunum of the patient; and
a hub having a plurality of openings, a first opening communicating with said first tubular member, and a second opening communicating with said second tubular member.

17. The assembly of claim 16, further comprising a closure member sized and arranged for selectively sealing said second opening.

18. The assembly of claim 16, wherein said first tubular member comprises a lumen extending therethrough, said assembly further comprising an elongated member selectively receivable in said lumen and removable therefrom.

19. The assembly of claim 16, wherein said hub further comprises a housing member extendable into said stoma, said first and second tubular members extending through said housing member.

20. The assembly of claim 16, wherein each of said first and second tubular members includes a lumen extending therethrough, and wherein at least said second tubular member includes a side port communicating with said lumen.
